(19) United States
(12) Patent Application Publication
(10) Pub. No.: US 2008/0199507 A1
(43) Pub. Date: Aug. 21, 2008

(54) Therapeutic Polymeric Pouch

(75) Inventors: Gary A. Skarja, Toronto (CA); Rebecca K. Ho, Toronto (CA); Michael H. May, Brantford (CA)

Correspondence Address: BORDEN LADNER GERVAIS LLP;Anne Kinsman, WORLD EXCHANGE PLAZA, 100 QUEEN STREET SUITE 1100, OTTAWA, ON, K1P 1J9, omitted

(73) Assignee: RIMON THERAPEUTICS LTD., Toronto, ON (CA)

(21) Appl. No.: 11/917,828

(22) PCT Filed: Jun. 19, 2006
(86) PCT No.: PCT/CA2006/000995

§ 371 Date: Dec. 17, 2007

Related U.S. Application Data
(60) Provisional application No. 60/691,257, filed on Jun. 17, 2005.

(51) Int. Cl. A61L 27/54; A61L 27/56

(52) U.S. Cl. 424/423

(57) ABSTRACT

An implantable or surface-applied device comprising a porous polymeric pouch containing a therapeutic polymer sealed in the pouch, to provide a localized therapeutic effect at the site of the application. The pores in the polymeric pouch being smaller than the therapeutic polymer, allowing body fluid to enter and exit the pouch and interact with the therapeutic polymer in the pouch in use, but not permitting the therapeutic polymer to leave the pouch.

### FIELD OF THE INVENTION

The present invention relates to an implantable or surface-applied medical device for delivering localized therapeutic action.

### BACKGROUND OF THE INVENTION

Most commonly, therapeutics are administered systemically via injection or ingestion leading to distribution throughout the patient's body. This method of administration is simple and effective for many situations, but can produce unwanted side-effects and limits dosage due to secondary effects. Therefore, local administration is often more effective in providing a site-specific therapeutic effect while reducing secondary, systemic complications. This has led to the development of a number of controlled release drug devices that can be implanted at the site of need. Though these devices typically increase local concentration of a drug, soluble drugs may diffuse or be transported from the application site resulting in some systemic distribution throughout the body.

The Applicant has previously described the use of chemically functionalised solid polymers that are capable of specifically interacting with biological components to effect a desired therapeutic outcome, such as new blood vessel formation (U.S. Pat. No. 6,261,585 “Generating Blood Vessels with Angiogenic Material Containing a Biocompatible Polymer and Polymerizable Compound” to Sefton et al., and U.S. Pat. No. 6,641,832 “Increasing Blood Flow to Tissue with Angiogenic Material Containing Polymer and Vascularizing Compound” to Sefton et al.), reduced connective tissue destruction (US Publ. 2004/0213758 “Hydroxamate-containing materials for the inhibition of matrix metalloproteinases” to Sefton et al.), and reduced bacterial colonization (WO 2004/090004 “Ancient Defense Polymer” to May et al.), all of which references are incorporated by reference herein. These “therapeutic polymers” may be used as localized therapeutics since they are insoluble and thus are not easily cleared or removed from the site of application by natural, physiological processes. Since these therapeutic polymers generally act through surface-mediated mechanisms, their degree of effect may be altered by varying their geometry. In particular, increasing solid polymer surface area by manufacturing them as small particles (<1 mm diameter) and/or highly porous solids can increase their biological activity on a mass-delivered basis. However, such geometries can make application and, more importantly, removal from the site more difficult; since small particles may be dispersed from or lodged in the tissue at the application site or engulfed by phagocytes leading to potential chronic or systemic complications. Also, non-porous solid pieces (e.g. films, pads) of weak cohesive strength (e.g. weak gels) may fragment during use creating small particles that can disperse and initiate chronic or systemic complications. In addition, sufficiently porous materials may become ingrown with host tissue making removal difficult, which again may lead to chronic side-effects. Therefore, there is a need for a means of enclosing such polymers.

Pouch medical devices have been described previously, including a mesh-reinforced porous foam containing insulin-producing cells (US 2004/0197374 “Implantable Pouch Seeded with Insulin-Producing Cells to Treat Diabetes” to Rezania et al.), and a pouch reservoir for the delivery of a therapeutic agent between the scalp and cranium (US 2004/0176750 “Implantable Reservoir and System for Delivery of a Therapeutic Agent” to Nelson and Truwit). However, in both these cases, the therapeutic reagent which is present or produced in the pouch is released into the surrounding tissue or body fluid. Olson et al. in US Patent Appl. 2004/0249382 “Tactical Detachable Anatomic Containment Device and Therapeutic Treatment System” describe a containment device for containing implanted bone cement in bones. In this latter case, the “pouch” is a barrier which prevents the bone cement from migrating in the body; the bone cement itself has merely a structural effect, but does not interact on a biological or biochemical level with the surrounding bone.

Thus, there is a need for a means of enclosing polymers to prevent their migration in the body but which also allows the polymers to interact with the body to achieve their therapeutic effect.

### SUMMARY OF THE INVENTION

It is an object of the present invention to provide an implantable or surface-applied porous pouch that contains therapeutic polymer(s) to provide a localized therapeutic effect at the site of application.

Thus, in one aspect the present invention provides an implantable or surface-applied device, comprising: a porous polymeric pouch and a therapeutic polymer sealed in the pouch. The pores in the polymeric pouch are smaller than the therapeutic polymer. This allows body fluid to enter and exit the pouch and interact with the therapeutic polymers in the pouch, but does not permit the therapeutic polymer to leave the pouch. The polymeric pouch comprises a porous polymer.

The device can act locally through the interaction of interstitial or extracellular bodily fluids with the enclosed polymer, which can preferentially bind or sequester targeted biological factors or cells to produce a therapeutic outcome. The pouch facilitates the easy application and removal of the active polymer(s), preventing dispersion from the site of application.

Advantageously, the pouches of the present invention provide a therapeutic effect by interaction of the contained polymer(s) with the local bodily fluid at the site of application resulting in an altered composition of the fluid rather than through the release of a therapeutic compound into the site of application. For instance, the therapeutic polymers may bind components of the bodily fluid, thereby removing or stabilizing them.

In another aspect, the present invention provides a method of providing a site-specific therapeutic effect. The method comprises implanting or surface-applying a device, having a porous polymeric pouch and a therapeutic polymer sealed in the pouch. The pores in the polymeric pouch are smaller than the therapeutic polymer, allowing body fluid to enter and exit the pouch and interact with the therapeutic polymers in the pouch, but do not permit the therapeutic polymer to leave the pouch.

In another aspect, the present invention provides a method for the delivery and removal of a therapeutic polymer to an animal, comprising applying the device herein described to a desired site in the animal; allowing the therapeutic polymer to exert its action; and removing the porous polymeric pouch when treatment is completed.

Other objects of the present invention will become apparent to those ordinarily skilled in the art upon review of the following description of specific embodiments of the invention.

### BRIEF DESCRIPTION OF THE DRAWINGS

FIG. 1 is a photograph of a porous, polymeric pouch containing 150-250 μm (microns) pro-angiogenic beads. The pouch is made of nylon with a mesh opening of 70 μm (microns).

FIG. 2 is a photograph of a porous, polymeric pouch (MI-Sorb™ dressing) containing matrix metalloproteinase (MMP) inhibiting polymer beads that has been heat sealed. The pouch is made of a medical-grade polyamide (MEDIFAB™) with a mesh opening of 36 μm (microns).

FIG. 3 is a schematic showing a method for producing multiple pouches ready for polymer bead filling.

FIG. 4 is a graph showing the effect of gamma radiation sterilization (28.6 kGy dose) on pouch seam tearing load.

FIG. 5 is a graph showing the effect of storage time on pouch seam tearing load.

FIG. 6 is a graph showing the effect of storage time on polymer bead MMP inhibitory activity.

FIG. 7 is a photograph of a pouch comprising a porous mesh layer and a non-porous polymer film layer. FIG. 7A shows the mesh side and FIG. 7B shows the non-porous side.

FIG. 8 is a graph showing the chlorhexidine release profile with time for chlorhexidine-loaded polymer beads.

### DETAILED DESCRIPTION

Generally, the present invention provides a medical device comprised of a porous, polymeric pouch containing a therapeutic polymer(s) that can be used for localized therapy through the interaction of the active polymer interior with components of bodily fluids. The use of a porous pouch allows for the interaction of the active polymer with bodily fluids at the site of application and facilitates the simple application and removal of the device. A variety of polymeric pouch materials and therapeutic polymers are contemplated.

### The Polymer Pouch

The polymeric pouch should have sufficient mechanical strength to resist breakage during handling (i.e. application and removal) and resist the stresses present at the application site. It is preferably made of a flexible and conformable material to allow for easy application and placement in irregularly shaped spaces. It should be generally non-adhesive to the tissue present at the site of application to facilitate easy removal.

The polymer that composes the enclosing porous pouch is a biocompatible polymer. Biocompatible polymers are defined herein as polymers that induce, when implanted, an appropriate host response given the application. For the purposes herein, they are essentially non-toxic, non-inflammatory, non-immunogenic, and non-carcinogenic. The polymer should also be biostable.

Furthermore, the polymer must be sufficiently hydrophilic to wet and allow aqueous bodily solutions to imbibe into it and pass through the pores present in it. In general, any polymer that exhibits a water contact angle of less than 90° will spontaneously draw aqueous solutions into its pores and allow for the passage of the solution through it. (Water contact angle is a quantitative measure of the wetting of a solid by water. It is the angle formed by the water at the three phase boundary where the water, gas (air), and solid intersect.) This is important, because the device exerts a therapeutic effect by the interaction of the enclosed therapeutic polymer(s) with components present in bodily fluids. Examples of suitable polymers for use as the porous pouch material include polyamides (i.e. nylon), polyesters, polyurethanes, polyacrylates, as well as surface-treated polyolefins.

The pouch polymer must be formable into an open-pored structure to allow for the ready passage of bodily solutions through it. The enclosing pores should be sufficiently small to prevent the escape of the enclosed active polymer(s) while permitting fluid passage in and out of the pouch. The pore size may be variable or uniform but must be smaller than the active polymer pieces contained within the pouch. For example, the pore size of the pouch may be less than or equal to about 50% of the size of the therapeutic polymer. For example, the active polymer may be fabricated into microspheres of variable size down to 10 μm (microns) diameter; in this case, the pore size should be less than 10 μm (microns), such as 5 μm (microns). However, if the enclosed polymer pieces are larger, the pore size of the pouch may range up to several millimetres. For example the porous polymeric pouch may have a pore size of about 5 microns to about 45 microns, or about 5 microns to about 4 millimetres.

The porous pouch may be fabricated as a felt, weave, sponge, expanded mesh, etc. using commonly employed industrial practices.

In one aspect, the pouch casing material may be a synthetic polymeric mesh (e.g. nylon, polyester) with well-defined pore size.

It is desirable that the pouch not be subject to ingrowth by the tissue surrounding the pouch. One way to help prevent ingrowth in cases where ingrowth could be a problem (i.e. the host tissue grows into the pouch, making removal difficult), the pore size of the pouch should be sufficiently small, i.e. smaller than the pore size of the therapeutic polymer inside, to make ingrowth unlikely. For example the pore size of the pouch may be less than 25 microns, when the pore size of the therapeutic polymer is at least 25 microns; the pore size of the pouch may be about 45 microns or less, when the pore size of the therapeutic polymer is more than 45 microns; the pore size of the pouch may be about 36 microns, when the pore size of the therapeutic polymer is at least about 37 microns. However, adherency also depends on the length of time the pouch is left in place at the site of application. The longer it is left in place, the smaller the pore sizes of the pouch need to be to make the pouch be non-adherent.

The pouch casing material may be made up entirely of porous polymer or only in part. It may be desirable, for instance, to have only one side or only the central area of one or both sides of the pouch made from the porous polymer. For instance, the pouch may comprise one side which is the porous polymer and the other side which is a non-porous barrier layer. For instance the barrier layer may be a non-porous polyethylene-polyester composite film, a moisture-resistant polyethylene film, a polyurethane film, a silicone polymer film, or a polyacrylate film. Such barrier layers are known in the art, and may be used, for example, to prevent dirt, dust, and moisture from entering the site of application, such as a wound.

### Therapeutic Polymer

By “therapeutic polymer”, it is meant a polymer which has a therapeutic effect (i.e. for treatment of a disease or condition) on the body. The therapeutic polymer must be capable of exerting a desired therapeutic effect upon application, through the interaction with bodily fluids, or components of the fluids, at the site of application. The therapeutic polymer should achieve the therapeutic effect through a biological and/or biochemical interaction with the bodily fluids and/or components thereof.

Examples of polymers which are suitable include, but are not limited to:
Polymers that are able to sequester matrix metalloproteases (MMPs) from extracellular fluid, thus reducing local tissue destruction. Polymeric beads (MMP-inhibiting beads) containing hydroxamate (HX) groups have been described which inhibit matrix metalloproteinases (MMPs) in US 2004/021378 A1. Such polymeric beads may be prepared, for instance, by surface modification of cross-linked polymethacrylic acid-co-methyl methacrylate beads to contain hydroxamate groups.
Polymers that are able to bind and act as a sink for soluble pro-angiogenic cytokines, leading to localized tissue vascularization. Polymeric beads (angiogenic beads) with angiogenic properties have been described in U.S. Pat. No. 6,261,585, U.S. Pat. No. 6,641,832, and US 2002/0037308 A1. Such beads comprise an angiogenic material consisting of a biocompatible polymer and a vascularizing compound. The vascularizing compound preferably consists of polymerizable compounds capable of forming anions and which promote the growth of blood vessels in their immediate vicinity and induces minimal or no fibrous capsule formation in the body. Examples of such vascularizing compounds include polymerizable compounds containing an ionizable group consisting of sulfates, sulfonic acid groups, or carboxyl groups. Examples of such polymerizable compounds include acrylic acid, methacrylic acid, crotonic acid, itaconic acid, vinylsulfonic acid, and vinylacetic acid, particularly methacrylic acid. The polymerizable compound preferably consists of methacrylic acid which is incorporated into the biocompatible polymer at the time of polymerization said biocompatible polymer is preferably a polyacrylate.
Other examples include those described in WO 04/90004 entitled “Ancient Defense Polymer” and PCT CA2006/000533 entitled “Pro-Angiogenic Scaffolds”, both of which are incorporated by reference herein. Such ancient defense polymers have antimicrobial activity, and comprise one or more discrete hydrophobic segments and one or more hydrophilic segments containing cationic functionality. Said hydrophobic segment may comprise, for example, 1) polymerized hydrophobic chain growth monomers; 2) polymerized step-growth monomers; or 3) hydrophobic (di)functional oligomers or polymers. Said hydrophilic segment may comprise, for example, 1) polymerized cationic chain growth monomers; 2) a polymer made from a mixture of cationic chain growth monomers and (i) uncharged monomers that are hydrophilic or (ii) hydrophobic monomers; or 3) cationic (di)functional oligomers or polymers. For instance, the hydrophobic segment may comprise polymerized hydrophobic alkyl methacrylates, aryl methacrylates, alkyl methacrylamides, or aryl methacrylamides. For instance, the hydrophilic segment may comprise polymerized methacrylates and/or methacrylamides. The ancient defense polymer may be a copolymer of 3-aminopropyl methacrylamide (AMA,) and poly(propylene oxide)monomethacrylate (PPO-ME). The ancient defense polymer may be a terpolymer of 3-aminopropyl methacrylamide (AMA,), poly(propylene oxide)monomethacrylate (PPO-ME), and methyl methacrylate.

The therapeutic polymers contained within the porous pouch may be fabricated into a variety of geometries, such as a solid film or slab, microspheres or beads, fibers, or a porous solid piece.

Generally, the therapeutic polymers will be porous (i.e. have pores), but they may also be non-porous.

The geometry of the enclosed active polymer may be tailored to vary the biological effect. For example, a polymer may produce a biological effect through the surface binding of a soluble factor. In this case, increasing the total surface area of the enclosed active polymer will serve to increase the therapeutic activity of the device. Surface area per unit mass may be increased by forming the polymer into microspheres or other small particles, such as beads. Microspheres or particles may be generated by commonly used processes such as suspension polymerization, emulsion polymerization, particle precipitation, solvent evaporation in suspension, and milling or grinding.

The bead geometry allows easy mixing of discrete populations of polymers with distinct physical and therapeutic properties in a single pouch to produce of variety of effects. In addition, flexible and soft devices may be produced through the use of enclosed beads independent of the bead physical properties since they can easily move in relation to each other. However, the use of beads requires relatively stringent pouch sealing and pore size requirements to prevent escape of the enclosed polymer.

Larger pieces (i.e. films, slabs) of the therapeutic polymer are more easily handled and enclosed than beads while retaining high surface area (if made porous) for biological interaction. However, the chemical composition of the polymers that comprise these larger pieces must be designed to provide the appropriate physical characteristics (e.g. flexibility, absorptivity).

In addition, introduction of pores into the therapeutic polymer increases surface area available for interaction with components of bodily fluids. Pores may be introduced in a number of ways, including: solvent casting with a porogen, phase inversion, foaming, fiber formation, meshing, freeze-drying etc.

The size of the therapeutic polymer should be larger than the pore size of the polymeric pouch to prevent the therapeutic polymer from escaping from the pouch. In the case where the therapeutic polymer is present as beads, this means that the size of the pores in the polymeric pouch must be smaller than the diameter of the beads. In the case, where the beads are of various sizes, the size of the pores in the polymeric pouch must be smaller than the diameter of the smallest beads. Preferably, the size of the pores must be no larger than half (50%) the diameter of the beads, preferably no larger than half (50%) of the diameter of the smallest beads in cases where the beads are of various sizes.

For other geometries, the pores in the polymeric pouch are preferably smaller than the shortest linear dimension of the polymer particles/pieces. Preferably the size of the pores in the pouch must be no larger than half (50%) the shortest linear dimension of the particles/pieces.

The degree of biological effect may also be modulated by varying the amount of polymer enclosed in the pouch.

The therapeutic polymer(s) contained in the pouch may also have some absorptive capacity that can be useful for particular applications, such as wound dressings. In this way, a device that is able to absorb excess wound fluid as well as provide a therapeutic benefit is possible. The polymers may be made absorptive by a number of techniques, including: inclusion of hydrophilic groups in the component chemistry, ionization of ionisable groups present in the polymer, alteration of crosslink density and design of the physical form (e.g. introduction of porosity). In other instances, the active polymer(s) may be either non-absorptive (through introduction of hydrophobic groups in the component chemistry, increased crosslink density and/or lack of porosity) or pre-swollen to provide a device that does not remove fluid from the site of application, but does provide a therapeutic effect. Use of a dry form of the therapeutic polymer, i.e. a form that possesses the ability to swell significantly on exposure to aqueous solutions, can render the device absorptive.

In order to provide multiple therapeutic effects, different therapeutic polymers, having a therapeutic effect, may be contained together within the porous pouch. The additional therapeutic polymers may be loose in the pouch, or they may be bound to or coated onto the pouch or bound to the other therapeutic polymers. The manner of incorporation in the pouch depends on the intended use of the pouch, and the properties of the additional therapeutic polymer(s). For instance, the anti-microbial polymers described in WO 04/90004 entitled “Ancient Defense Polymer” may be coated directly onto the pouch to provide anti-microbial properties to the pouch.

Another way to provide multiple therapeutic effects is to use two or more pouches in succession. Pouches containing one type of therapeutic polymer may be initially applied and removed; subsequently pouches containing a different active polymer can be applied to direct a desired effect. For example, pouches containing an antimicrobial polymer may be applied initially to a wound site, followed by pouches containing a pro-angiogenic polymer to assist subsequent healing.

### Additional Therapeutic Components

The pouch may additionally include other therapeutic non-polymeric components. Such therapeutic components may include numerous therapeutic compounds known in the art, such as anti-infective compounds. Anti-infective compounds include antibiotics and antiseptics. Antibiotics include, for example, aminoglycosides (e.g. gentamicin, neomycin), tetracyclines, penicillins (e.g. ampicillin, amoxicillin), carbapenems, fluoroquinolones (e.g. ciprofloxacin), macrolides, and antimicrobial peptides or derivatives thereof. Antiseptics are chemical agents that are potentially toxic to both microbial cells and host cells; therefore, their use is limited to topical application on wounds and intact skin. Examples of antiseptics include: biguanides (e.g. chlorhexidine), quaternary ammonium compounds, heavy metal derivatives (e.g. silver), and iodine.

The additional therapeutic compounds may be loose in the pouch they may be coated on or bound to the therapeutic polymer(s), or they may be bound to or coated onto the pouch. The manner of incorporation in the pouch depends on the intended use of the pouch, and the properties of the additional biologically active compound(s).

For instance, anti-infective compounds, such as chlorhexidine, may be loaded into/onto beads of the therapeutic polymer. For instance, they may be coated/loaded onto beads of polymers that are able to sequester MMPs from extracellular fluid (i.e. MMP-inhibiting beads, described in US 2004/0213758). Other types of beads that may also be treated in a similar manner with an anti-infective compound (e.g. chlorhexidine) include beads of the polymers that are able to bind and act as a sink for soluble pro-angiogenic cytokines (i.e. angiogenic beads, described in U.S. Pat. No. 6,261,585, U.S. Pat. No. 6,641,832, and US 2002/0037308 A1).

### Methods of Making the Device

The principal steps in the fabrication of the device are creation of the porous pouch and pouch filling with the therapeutic polymer. These processes may be carried out separately or together depending on the nature of the filling polymer. For example, a solid film of filler polymer may be laid between two sheets of polymer mesh and the mesh sealed around it to create the device. In contrast, an open pouch may be fabricated first by sealing three sides and the filler polymer may be added in the form of particles or a solid piece followed by a final sealing to close the pouch.

The pouch may be sealed by a number of techniques such as heat sealing, adhesive, stitching, welding and folding. These are known in the art. The form of the active polymer enclosed in the pouch and the site of application dictates the requirements of the sealing method. The seals must prevent the loss of the contained polymer during application, use, and removal through leakage and/or breakage.

### Therapeutic Applications

The pouch in accordance with this invention may have a number of applications where localized interaction with a therapeutic polymer is desired. Such applications may include: chronic wound healing, treatment of degenerative joint disease, prevention of tumor progression, aneurysm prevention, treatment of local ischemia and topical treatment of infection or bacterial colonization.

### EXAMPLES

### Example 1

### Formation of Filled Pouches—Chronic Wound Dressings

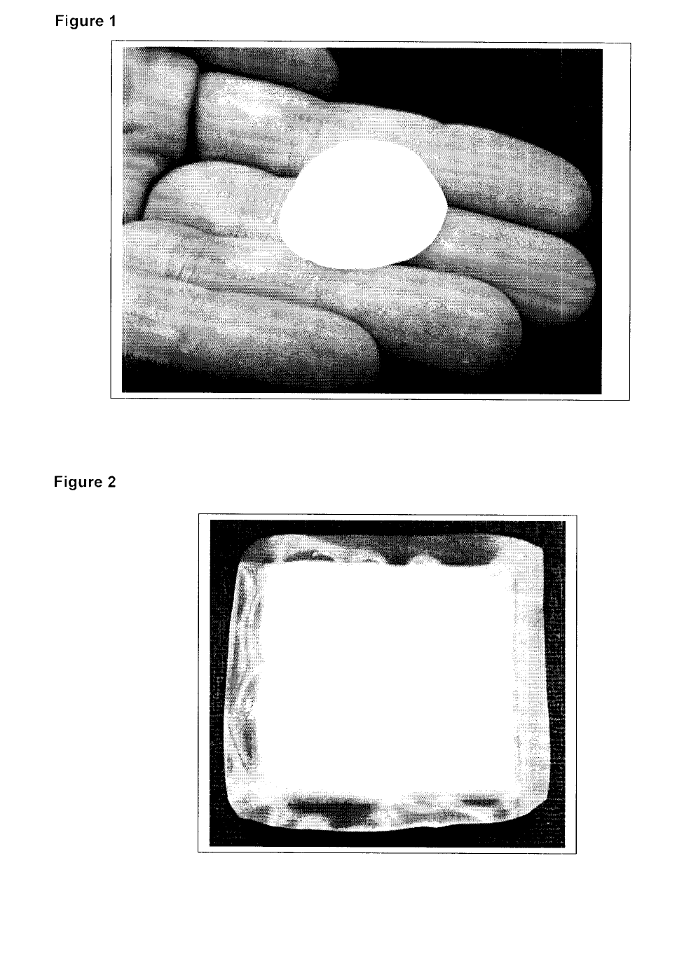

A number of pouches containing matrix metalloproteinase (MMP) inhibiting polymeric beads (100-250 μm diameter) were produced for use as a wound dressing useful for non-healing skin wounds (referred to as MI-Sorb™ Dressings). Prototype dressing pouches were produced by both heat sealing and use of an adhesive and both a nylon and polyester mesh (FIG. 1). Final dressing pouch dimensions of approximately 2.5×2.5 cm were selected and 700 mg of dry, polymeric beads were added to the pouch (FIG. 2). The mesh pouches could not be completely filled with dry beads since they swell significantly in moist environments and overfilling may result in rupture during use. The pouch mesh selected was a medical-grade polyamide mesh purchased from SEFAR Filtration Inc. (Buffalo, N.Y.). The mesh (MEDIFAB™, 36 μm (microns) mesh opening) is a precision monofilament fabric that is produced from raw materials that comply with the Code of Federal regulations (21CFR177) and European guidelines (BGA, EU-directives), and is fabricated in ISO 9001 certified facilities that follow applicable GMP guidelines. The material is non-hemolytic, non-cytotoxic, has low extractables and endotoxin content, and passes USP plastics class VI/ISO 10993 tests. A mesh opening size of 36 μm (microns) was chosen to ensure complete bead retention in the pouch while allowing easy exudate fluid transfer and interaction with the enclosed polymeric beads.

Heat sealing was used for mesh pouch fabrication and dressing sealing. This rapid and effective method avoids the introduction of any additional materials (i.e. adhesives) to the dressing that may modify the effect of the beads or produce unwanted side-effects. A 16″ impulse heat sealer (American International Electric Inc., 5 mm seal width) was used to produce the sealed dressings. The heat sealing conditions (heat sealer setting, time heating applied and time elapsed between sealing) for the MEDIFAB™ mesh were investigated and standardized leading to an optimal procedure to produce consistently good seals.

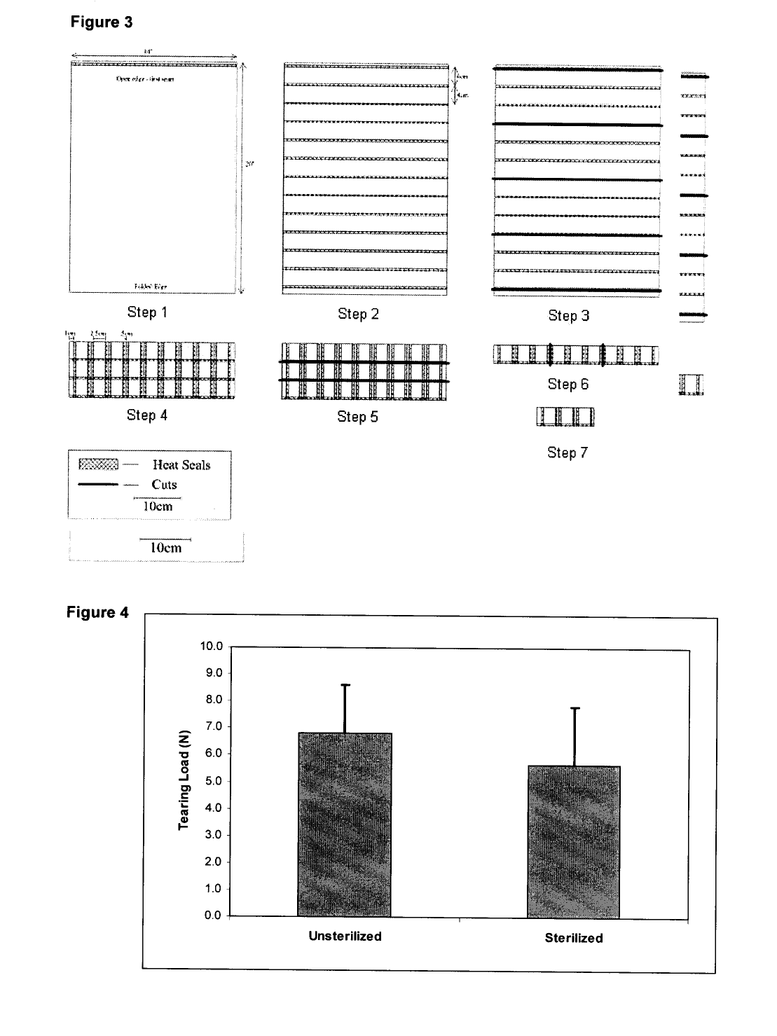

Next, a method for producing mesh pouches quickly was developed and tested. The MEDIFAB™ mesh was received as a 40″ wide roll. Therefore, a method was developed that involved cutting a 14″ strip from the 40″ roll, folding it in half and sealing the two layers together creating a 20″ long×14″ wide two layered piece. This piece was marked and heat seals were applied creating over 100 empty mesh bags from each piece (FIG. 3). A simple, accurate and reliable method for polymeric bead filling of the mesh bags was developed to produce a large number of dressings quickly. Metered volumes of the beads may be added through small funnels. However, this method was found to be quite laborious and time-consuming. Instead, a simpler method was adopted that consisted of scooping a defined volume of beads using a measuring spoon, leveling the scooped volume of beads with a straight-edge and transferring the beads to a mesh bag using the spoon. It was determined that a leveled ¼ teaspoon reliably and rapidly transferred approximately 700 mg of beads with good precision (±5%) by this method.

### Example 2

### Characterization of Filled Pouches

MI-Sorb™ dressings fabricated as described in Example 1 were characterized for pouch seam integrity, amount of filled beads, effect of gamma radiation sterilization and effect of storage time using a variety of test methods.

Pouch seam tear testing was performed on 100 samples of heat sealed MEDIFAB™ mesh using a modified ASTM 180° peel test (ASTM F-88). Briefly, the pouch samples (1 cm wide×5 cm long) were tested on an Instron 8501 Testing Machine using a 100 N load cell and a separation rate of 300 mm/min. The load required to rupture the seam area was recorded to produce an accurate estimate of the average seam strength and an acceptable minimum strength. In addition, the effect of modifying the heating time required to generate a seal and the inclusion of polymer beads in the seam on the seam strength was investigated. In general, the strength of the heat sealed seam was not sensitive to the heating time applied since little difference in tearing loads was observed. Underheating during seam formation did result in a modest reduction in tearing load (from 7 N to 4.9 N) but the seams were still complete. More significantly, inclusion of a moderate to large number of beads in the seam did result in diminished tearing loads (from 7 N to 1.9-2.5 N) that were below the acceptance value (3 N). Thus, during the subsequent production of MI-Sorb™ dressings, the seams were visually inspected to ensure that dressings did not contain substantial numbers of beads in the seams which could lead to device rupture or escape of beads.

Bursting force measurements were also made to determine the compressive load that the fluid-swollen dressings are capable of withstanding. Sample dressings were incubated in phosphate-buffered saline (PBS, pH 7.4) at room temperature for at least 2 h to completely swell the beads. The fluid-swollen dressings were tested for compressive load at break in an Instron 8501 Testing Machine using a plate attachment and a compression rate of 1 mm/min. The dressing break point was determined visually and the compressive load at break was recorded. The required to rupture the dressings was determined to be greater than 3000 N. Since the walking load generated by a person is generally estimated at 1.2 to 1.4 times body mass, a 3000 N force is equivalent to the walking force generated by a 480 lb individual.

The mass of beads contained within the pouches was assessed by cutting open and pouring out the beads from 37 MI-Sorb™ Dressings produced, packaged and sterilized in a initial pilot run (fabricated at Rimon, sterilized by Steris-Isomedix). The average bead mass contained within the dressings was 704 mg±34 mg. Therefore, the filling technique described in Example 1 was effective at delivering the desired dose of beads to the pouch with a relatively high level of precision.

The MI-Sorb™ Dressings were individually packaged for sterilization in Tyvek™/Polyester-polyethylene laminate two layer pouches joined by 10 mm wide chevron adhesive seams (Tolas Health Care Packaging, Feasterville Pa.) that were sealed after addition of the dressing with a 10 mm wide heat seal (in accordance with FDA sterile packaging guidelines). Sterilization was done using gamma radiation at a minimum dose of 25 kGy (considered a maximum dose for medical device sterilization). Dressing material properties were examined by pre- and post-sterilization testing of both the beads and mesh. Dressings were fabricated, packaged, labeled and sent to Steris-Isomedix (Whitby, ON) for radiation sterilization (received an average radiation dose of 31.6 kGy). After sterilization, the mesh seam tear strength was unchanged indicating that the physical integrity of the mesh was not negatively affected by the irradiation procedure (FIG. 4). In addition, the MMP inhibitory capacity of the beads was determined using a FITC-gelatin assay pre- and post-sterilization. Briefly, the bead effect on MMP activity was determined as follows: the beads were incubated for 1.5 h in an MMP-2 solution (4 U/mL), the solution was removed, the solution pH adjusted to 7.4, a gelatin-fluorescein conjugate solution was added and the rate of fluorescence generation was measured. The initial rate of increase of fluorescence (RFU/min) was taken as a measure of solution MMP activity. Percent reductions in MMP activity (MMP inhibitory activity) subsequent to bead incubation were determined in comparison to control MMP-2 solutions not exposed to the polymer beads. A modest reduction in MMP inhibitory activity (˜8%) was detected subsequent to radiation sterilization.

To confirm the sterility of the gamma irradiated dressings, a USP standard method was performed that involves incubating the sterilized dressing in sterile media for 2 weeks and assessing for any bacterial growth in the media. This test demonstrated no bacterial growth for several batches of gamma irradiated dressings, which informally validates the sterilization method and indicates that all dressings tested were adequately sterilized.

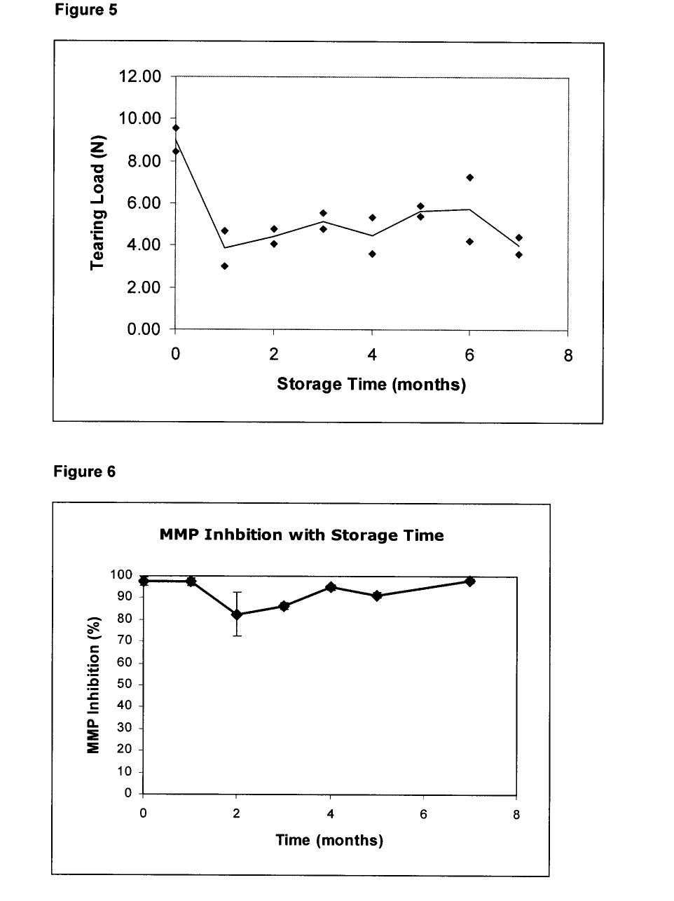

Finally, the effect of storage time on MI-Sorb™ dressing properties was investigated. Both the pouch seam strength (breaking load) and bead MMP inhibition (FITC-gelatin assay) were determined once a month for 6 months post-sterilization. FIGS. 5 and 6 shows the results of this analysis indicating no significant change in either pouch seam strength or bead MMP inhibitory activity on storage. This data establishes a minimum shelf-life for the dressing of 6 months.

### Example 3

### Composite Pouch Formation

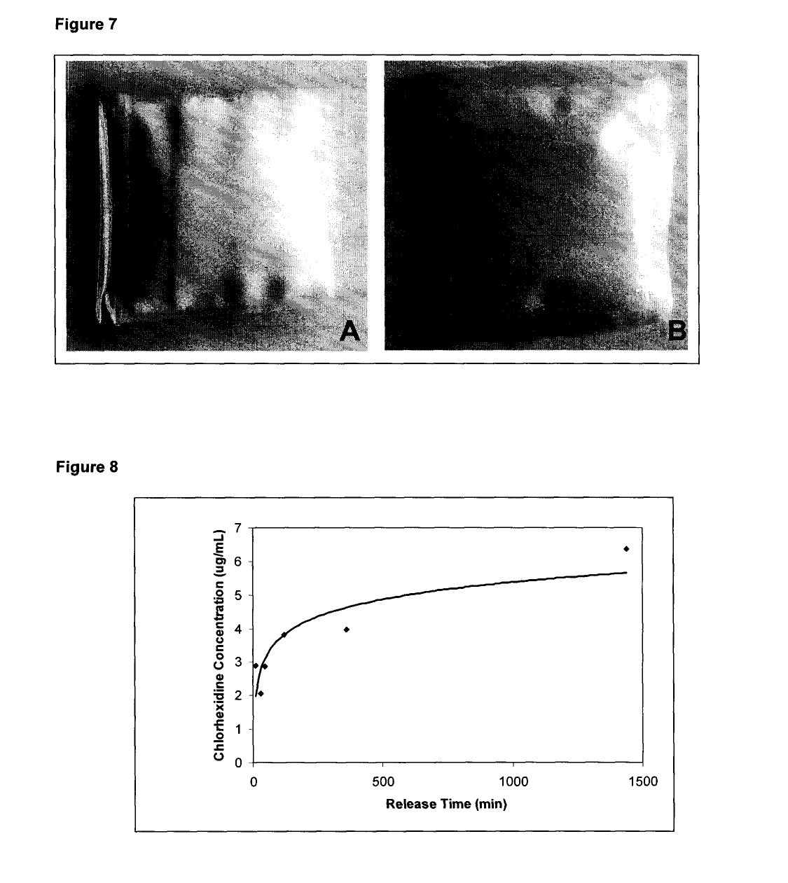

In addition to the mesh pouches (shown in FIGS. 1 and 2) that comprise two mesh layers joined together, additional prototype pouches were produced that consist of one porous mesh layer joined to a non-porous polymer film layer (FIG. 7). This pouch consists of the MEDIFAB™ mesh heat sealed to a non-porous polyethylene-polyester composite film containing MMP inhibiting beads. The non-porous layer of this pouch may provide a barrier layer that is necessary in skin wound dressings to provide excessive moisture loss and bacterial infection while the porous mesh layer allows easy fluid transport to the enclosed beads thereby facilitating the therapeutic effect.

### Example 4

### Formation of MMP-Inhibiting Polymeric Beads

MMP inhibiting polymer beads were produced through the introduction of hydroxamate functional groups to methacrylic acid-containing copolymers. HX (hydroxamate, i.e. —C(═O)N(OH)H) polymer was synthesized by surface modification of cross-linked polymethacrylic acid (PMAA)-co-methyl methacrylate (MAA) beads (resulting in a novel composition of PMAA-MMA-HX). Crosslinked poly(methyl methacrylate-co-methacrylic acid) (PMMA-MAA) beads were suspended in a suitable organic solvent (e.g. DMF, THF, diethyl ether) at approximately 10% wt/vol and allowed to equilibrate in solvent for at least 30 min at 0° C. while stirring. A 100% molar excess of N-methyl morpholine and chloroformate, relative to the MAA content of the beads, was added to the bead suspension. The reaction proceeded at 0° C. for 30 min. The beads were filtered from suspension and washed with DMF. The beads were transferred to a vessel containing a 100% molar excess of hydroxylamine solution in water and the reaction proceeded at ambient temperature for at least 1 hour. The beads were then filtered and washed with water, 0.1 M HCl, again with water, dried at 55-60° C. and sieved to the desired size range (100-250 μm (microns) diameter).

The dried beads were further purified as follows:
1. Add 0.2 M NaOH (10 mL/g of beads). Soak for >6 hours.
2. Decant supernatant. Add Milli-Q™ water (10 mL/g of beads). Stir and allow to settle.
3. Repeat Step 2.
4. Repeat Step 2. Allow to soak for >12 hours.
5. Decant supernatant. Add 0.3 M HCl (10 mUg of beads). Soak for >6 hours.
6. Repeat step 2.
7. Repeat step 2.
   The above is considered to be one (1) “washing cycle”. A total of six (6) washing cycles was performed to ensure purity. Once the washing was complete, the beads were filtered through #4 Whatman paper and rinsed with Milli-Q™ water (20 mUg of beads). Finally, the beads were dried at 60° C. under vacuum.

The hydroxamate content (as indicated by nitrogen content) of the copolymer beads may be varied in this process by altering the acid content of the base copolymer from 15 to 80 mol % MAA. However, the most commonly used composition for the MMP inhibiting beads (i.e. enclosed in MI-Sorb™ Dressing) used in the pouches of the present invention was based on copolymers containing 62 to 66 mol % MAA.

### Example 5

### Antimicrobial Releasing Pouch

The MMP inhibiting polymer beads that may be contained within the pouch can also be rendered antibacterial by incubation with a common antibacterial compound, such as chlorhexidine. Chlorhexidine may be bound to the beads and subsequently released into the environment surrounding the application site of the pouch in a predictable way.

MMP inhibiting beads were loaded with chlorhexidine as follows. A chlorhexidine diacetate solution (1.5% v/v) in water made up and filtered using a 0.22 μm syringe filter. MMP inhibiting polymer beads were added to the chlorhexidine solution (1.5 g in 50 mL) and incubated with periodic vortexing for 24 h at room temperature. The beads were filtered from the chlorhexidine solution and dried at 60° C. under vacuum for at least 18 h.

Chlorhexidine release from the beads was performed as follows. The beads were weighed out into 1.5 mL microcentrifuge tubes and endotoxin-free water was added to each tube (100 mg beads in 1 mL water). After the desired incubation time (up to 24 h) the bead-containing microcentrifuge tubes were vortexed and 900 μL of the solution was removed and analyzed for chlorhexidine concentration by UV spectroscopy (absorbance read at 260 nm). Chlorhexidine concentration was quantified by comparison to a standard curve generated using known concentrations of chlorhexidine solutions. FIG. 8 shows a typical chlorhexidine release profile for the loaded beads indicating progressive release up to 24 h.

### Example 6

### Angiogenic Beads

Angiogenic beads were produced by a suspension copolymerization process. Crosslinked copolymers of methacrylic acid and methyl methacrylate were synthesized using the following procedure. Monomers and initiator were added to a reactor containing a CaCl₂/H₂O suspending solution with tricalcium phosphate (TCP) dispersing agent, with stirring. The reaction proceeded for 5 h at 70° C. under nitrogen. The heat was removed, allowing the reactor contents to cool to at least 50° C. Then a 2 M HCl solution was added to the reactor to dissolve the TCP and the beads were filtered from the reaction solution using Whatman 113 filter paper. The filtered beads were then washed by incubation in a series of aqueous and organic solvent solutions to remove extractable impurities. The purified beads were then dried at 60° C. under vacuum for at least 24 h and sorted into various size fractions by sieving. Beads containing 40-50 mol % MAA (150-250 μm (microns) diameter) were found to elicit the most favorable angiogenic response upon implantation in a variety of small animal models.

1. An implantable or surface-applied device, comprising:
a porous polymeric pouch, comprising a porous polymer; and
a therapeutic polymer sealed in the pouch;
wherein the pores in the polymeric pouch are smaller than the therapeutic polymer, allowing body fluid to enter and exit the pouch and interact with the therapeutic polymers in the pouch in use, but not permitting the therapeutic polymer to leave the pouch.

2. The device of claim 1, wherein the porous polymer is selected from the group consisting of: polyamides, polyesters, polyurethanes, polyacrylates, and surface-treated polyolefins, and mixtures thereof.

3. The device of claim 1 or 2, wherein the porous polymeric pouch comprises:
a bottom side comprising the porous polymer; and
a top side comprising a barrier layer.

4. The device of claim 1, 2, or 3, wherein the pore size of the porous polymer is about 5 to about 45 microns.

5. The device of any one of claims 1 to 4, wherein the porous polymer is non-adherent to tissue.

6. The device of any one of claims 1 to 5, wherein the therapeutic polymer is absorbent.

7. The device of any one of claims 1 to 6, wherein the therapeutic polymer contains a hydroxamate group.

8. The device of claim 7, wherein the therapeutic polymer is a polymethacrylic acid-co-methyl methacrylate bead, surface modified with hydroxamate groups, which binds a matrix metalloproteinase.

9. The device of any one of claims 1 to 6, wherein the therapeutic polymer is an angiogenic material.

10. The device of claim 9, wherein the angiogenic material consists of a biocompatible polymer and a vascularizing compound, said vascularizing compound consisting of polymerizable compounds capable of forming anions and which promote the growth of blood vessels, said polymerizable compounds being selected from the group consisting of acrylic acid, methacrylic acid, crotonic acid, itaconic acid, vinylsulfonic acid, and vinylacetic acid.

11. The device of claim 10, wherein said angiogenic material consists of a polyacrylate.

12. The device of any one of claims 1 to 11, wherein the therapeutic polymer is coated or bound to chlorhexidine.

13. The device of any one of claims 1 to 12, wherein the therapeutic polymer is a porous or non-porous bead.

14. The device of any one of claims 1 to 12, wherein the therapeutic polymer has a porous or non-porous monolithic geometry.

15. The device of any one of claims 1 to 14, wherein the pouch contains a mixture of two or more therapeutic polymers.

16. The device of claim 15, wherein the pouch contains:
an angiogenic polyacrylate bead, and
a polymethacrylic acid-co-methyl methacrylate bead, surface modified with hydroxamte groups, which binds a matrix metalloproteinase.

17. The device of any one of claims 1 to 16, wherein the polymeric pouch is coated with an anti-microbial polymer.

18. A method of providing a site-specific therapeutic effect, comprising implanting or surface-applying a device, comprising a porous polymeric pouch and a therapeutic polymer sealed in the pouch; wherein the pores in the polymeric pouch are smaller than the therapeutic polymer, allowing body fluid to enter and exit the pouch and interact with the therapeutic polymers in the pouch, but not permitting the therapeutic polymer to leave the pouch.

19. A method for the delivery and removal of a therapeutic polymer to an animal, comprising:
applying the device of any one of claims 1 to 17 to a desired site in the animal;
allowing the therapeutic polymer to exert its action; and
removing the porous polymeric pouch when treatment is completed.
